(19) United States
(12) Patent Application Publication
(10) Pub. No.: US 2023/0330024 A1
(43) Pub. Date: Oct. 19, 2023

(54) NOVEL TREATMENT REGIMEN OF CANCER

(71) Applicant: MOROXITE AB, TOLLARP (SE)

(72) Inventors: Lars LIDGREN, TOLLARP (SE); Magnus TÄGIL, TOLLARP (SE); Deepak RAINA, TOLLARP (SE); Liu YANG, TOLLARP (SE)

(21) Appl. No.: 18/247,740

(22) PCT Filed: Oct. 5, 2021
(86) PCT No.: PCT/EP2021/077420

§ 371 Date: Apr. 3, 2023

(30) Foreign Application Priority Data
Oct. 5, 2020 (SE) 2051170-5

(51) Int. Cl. A61P 35/00; A61K 9/14; A61K 31/675; A61K 31/704

(52) U.S. Cl. CPC A61K 9/143; A61K 31/675; A61K 31/704; A61P 35/00

(57) ABSTRACT

Present invention relates to a novel treatment regimen for cancer.

### TECHNICAL FIELD

Present invention relates to a novel composition and administration mode of a particulate material, such as e.g. micro and/or nano sized hydroxyapatite (HA) and an anthracycline such as e.g. doxorubicin and a bisphosphonate, such as e.g. zoledronic acid. Present invention relates to i.a. use of the novel compositions according to the invention for treatment of cancer and in particular solid tumours.

### BACKGROUND OF THE INVENTION

Both osteosarcoma and Ewing’s sarcoma are highly malignant tumours, usually affecting children and adolescents. Chemotherapy has dramatically increased the prognosis for the affected individuals both pre and postoperatively but for non-responders the options are few. For osteosarcoma, methotrexate, doxorubicin (Adriamycin), and cisplatin (MAP) form the backbone of standard treatment protocol. However, approximately 40% patients show a poor response to chemotherapy, with a 5-year survival of 45% to 55%. A recent clinical study conducted by EURAMOS-1 compared postoperative MAP with MAP plus ifosfamide and etoposide (MAPIE) in patients with a poor response. No significant benefit was achieved by using MAPIE and its administration was associated with increased toxicity without improving event-free survival. For Ewing’s sarcoma (ES), induction chemotherapy with vincristine, doxorubicin, and cyclophosphamide (VIDE) is now considered the standard of care in Europe, whereas compressed vincristine, doxorubicin, and cyclophosphamide plus ifosfamide and etoposide (VDC-IE) is the North American standard. In a study conducted by Italian Sarcoma Group (ISP) and Scandinavian Sarcoma Group (SSG), they showed 51% (154 out of 300) Ewing’s sarcoma patients had a poor response to chemotherapy. For poor response patients, five-year event-free survival (EFS) was about 33% under conventional chemotherapy. A review from E Hanafy et al. concluded that no strong evidence exists that intensifying systemic chemotherapy in poor responders results in better outcome or at least still needs further validation. Adding more systemic drugs does not improve outcome and rather, other strategies to efficiently deliver the drugs to the target sites are thus necessary.

Various ways of improving drug delivery to the target site have been explored within the last three decades, which can be divided into passive or active target delivery based on the mechanism. Passive target delivery is based on enhanced permeability and retention effect (EPR effect) by loading drugs on a nano-size carrier such as Doxil, for which doxorubicin (Dox) is encapsuled in liposome. However, limited evidence base to support the superiority of the liposomal doxorubicin compared to the conventional doxorubicin in efficacy when it comes to patients, with the only benefit to reduced cardiotoxicity. Active target delivery is trying to decorate surface of the nanocarrier with ligands binding to the receptors overexpressed on tumour cells, which has been considered to significantly increase the quantity of drug delivered to the target cell compared to free drug or passively targeted nano-systems. However, a comprehensive review paper from Stefan Wilhelm published on Nature Reviews Materials reported only 0.7% (median) of the administered nanoparticle dose is found to be delivered to a solid tumour. Another review paper from Kinam Park published on Journal of Controlled Release has the same opinion that only a very small fraction (<5%) of the total administered formulation is actually delivered to the intended target site. Moreover, the fabrication processes of these nano-systems are highly complicated regardless of passive or active strategy. Thus, nano particles as carrier for tumour drugs has been largely unsuccessful in clinical translation mainly due to insufficient carriers and delivery at the target site.

Hydroxyapatite (HA), a main component of calcified bone (70% inorganic mineral), has been successfully synthesized and approved for clinical usage. Recently, the inventors of present invention found doxorubicin (Dox) could bind to nano-size synthetic hydroxyapatite (HA). Functionalized nano HA could facilitate delivery of drugs intracellularly, which in turn could enhances the cytotoxic effect. Combining systemic Dox and zoledronic acid (ZA) together has indicated a synergistic anti-tumour effect on subcutaneous breast tumour in mice. Present invention relates i.a. to a completely novel composition and treatment regimen which entails combining Dox and ZA using a carrier containing nano HA for local delivery and showed increased killing of tumour cells. This presents a significant clinical step forward and a major contribution to the art which overcomes the above discussed shortcomings. The findings of these aspects will be further elaborated upon below and in the following text.

### SUMMARY OF THE INVENTION

Present invention relates to a novel composition comprising a particulate material. The particulate material may be in various size particle sizes such as e.g. in micro sized particles. In another aspect, the particles may be e.g. in nano sized particles. In yet a further aspect, the particulate material may comprise a mixture of micro size and nano sized particles. The particulate material may be e.g. hydroxyapatite (HA).

The invention further relates to one or more drugs commonly used in treatment of therapy of cancer or tumour related diseases. In one aspect, the invention relates to anthracyclines, which may be drugs extracted from Streptomyces spp. The anthracyclines may be e.g. doxorubicin, daunorubicin, epirubicin, aldoxorubicin, annamycin, plicamycin, pirarubicin, aclarubicin and idarubicin or any suitable mixtures thereof. Further examples are e.g. mitoxantrone or valrubicin.

However, it is in principle possible to use any compounds commonly used in the treatment of cancer (antineoplastic drugs) such as e.g. antimetabolites, antimitotics, antitumor antibiotics, asparagine-specific enzymes, any type of biosimilars, DNA-damaging agents (antineoplastics), alkylating agents, DNA-repair enzyme inhibitors, histone deacetylase inhibitors, hormones (e.g. corticosteroids), hypomethylating (demethylating) agents, immunomodulators, Janus-Associated Kinase (JAK) inhibitor, monoclonal antibodies, Phosphoinositide 3-kinase inhibitors (PI3K inhibitors), proteasome inhibitors, Selective Inhibitors of Nuclear Export (SINE), and tyrosine kinase inhibitors and the likes.

In another aspect, present invention relates to one or more compounds capable of binding to apatite in any binding mode. In one aspect, the invention relates to a combination of at least two compounds capable of binding to calcium apatite. In a further aspect, present invention relates to e.g. one or more bisphosphonate and e.g. one or more agents capable of binding to hydroxyl apatite, wherein such agents may be any drug used in cancer treatment under the proviso that such agent is capable of binding to hydroxyapatite.

The above mentioned compounds or agents may be used for treatment of various clinical indications such as e.g. acute lymphocytic leukaemia, acute myelogenous leukaemia, Hodgkin’s lymphoma, non-Hodgkin’s lymphoma, bladder cancer, rapidly recurrent (Ta or Ti) or in situ transitional cell carcinoma (intravesical therapy), neoadjuvant treatment, metastatic transitional cell bladder cancer, breast cancer, adjuvant therapy following axillary lymph node resection in primary breast cancer, metastatic breast cancer, other metastatic cancers, ovarian cancer, osteogenic sarcoma, ewing’s sarcoma, soft tissue sarcoma, thyroid cancer, neuroblastoma, Wilm’s tumor, small cell lung cancer. Other clinical conditions may be advanced endometrial carcinoma, metastatic hepatocellular cancer, multiple myeloma, advanced renal cell carcinoma (with sarcomatoid features), thymomas and thymic malignancies, uterine sarcoma, and Waldenstrom macroglobulinemia. Overall, present invention relates to treatment of any solid tumours. Non-limiting examples are sarcomas, carcinomas, and lymphomas.

Carcinomas are in general cancers derived from epithelial cells. This group includes many of the most common cancers that occur in older adults. Nearly all cancers developing in the breast, prostate, lung, pancreas, and colon are carcinomas. This the invention relates to treatment of cancers in tissue in breast, prostate, lung, pancreas, and colon.

Sarcomas are in general cancers arising from connective tissue (i.e. bone, cartilage, fat, nerve), each of which develop from cells originating in mesenchymal cells outside of the bone marrow. This the invention relates to treatment of cancers in connective tissue, i.e. in bone, cartilage, fat, nerve, etc.

Non-limiting examples of lymphomas are e.g. non-Hodgkin lymphoma (NHL) and Hodgkin lymphoma which present invention also relates to.

In another aspect, the invention relates to any compound, which comprises the chemical structure of

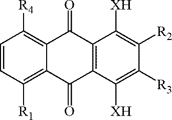

or any substitution pattern thereof including any pharmaceutically acceptable salts thereof. R₁ and R4 may be same or different and may be e.g. hydrogen, hydroxy or methoxy. R₂ and R₃ may be same or different and may be any suitable substitution (including hydrogen) and may together form any ring system. X may be same or different and may be selected from oxygen or nitrogen, such that X may be NR₅ and R₅ may be any suitable substituent.

In one aspect, present invention relates to treatment of any bone related tumour type such as e.g. osteosarcoma (OS) or osteogenic sarcoma (OGS).

Furthermore, the invention relates to one or more bisphosphonates such as e.g. such as e.g. zoledronic acid, etidronic acid, clodronic acid, tiludronic acid, pamidronic acid, neridronic acid, olpadronic acid, alendronic acid, ibandronic acid, risendronic acid or any pharmaceutically acceptable salts thereof. The bisphosphonate be also be a salt wherein a radioactive compound is present or a radioactive compound as ⁹⁹Tc or ²²³Ra or strontium or samarium.

In another aspect, the bisphosphonate may be e.g. any compound falling under the formula (Ia) or (Ib):

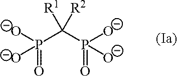

or any acceptable pharmaceutical salt thereof. In principle, R¹ and R² may be any suitable substituents and may be identical or different. Elements and compounds, may be referred to either by their full name or by their IUPAC name. Alternatively, a bisphosphonate may be represented by the formula:

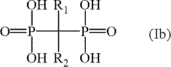

or any acceptable pharmaceutical salt thereof. In principle R¹ and R² may be any suitable substituents and may be identical or different.

Present invention also relates to a novel administration method.

In one aspect, the invention relates to administration wherein a subject in need of a treatment is administered the particulate material which may be e.g. HA (hydroxyapatite). Thereafter, the anthracyclines and bisphosphonate are administered to the subject in any order or simultaneously.

In another aspect, the particulate material onto which the one or more anthracyclines are absorbed, or otherwise bound to the particulate material, is administered to a subject. Said composition (HA+anthracyclines) is administered to a subject after which one or more bisphosphonates is/are administered.

In yet a further aspect, the particulate material onto which the one or more bisphosphonates are absorbed or otherwise bound to the particulate material is administered to a subject. Said composition (HA+bisphosphonates) is administered to a subject after which one or more anthracyclines is/are administered.

In yet another aspect, the particulate material onto which the one or more bisphosphonates and one or more anthracyclines are absorbed or otherwise bound to the particulate material and is subsequently administered to a subject.

The inventors of present invention have surprisingly found that the combinations of particulate material and bisphosphonates and anthracyclines result in an increased cell toxicity in respect of tumorous cells.

### BRIEF DESCRIPTION OF THE DRAWINGS

FIG. 1 illustrates the synergistic effect observed by the combination of particles with hydroxyapatite, doxorubicin and zoledronic acid with effect on cell viability observed at day 1, day 4 and day 7 respectively (after administration) in a head-to-head study of control (a control not containing any hydroxyapatite particles), nHA (nano sized particles of hydroxyapaptite), nHA-D (nano-sized particles of hydroxyapatite+doxorubicin), nHA+Z (nano-sized particles of hydroxyapatite+zoledronic acid), and nHA-D-Z (nano-sized particles of hydroxyapatite+doxorubicin+zoledronic acid).

FIG. 2 illustrates results from the MTT assay (assay employing enzymes that are capable of reducing the tetrazolium dye MTT 3-(4,5-dimethylthiazol-2-yl)-2,5-diphenyltetrazolium bromide to its insoluble formazan, which has a purple colour) showing the cytotoxic effect of DOX (doxorubicin) and ZA (zoledronic acid) functionalized nano-HA (hydroxyapatite) particles on A549 lung cancer cells. It is clearly illustrated that the DOX+ZA combination was the most toxic for the A549 cells.

FIG. 3 illustrates the tumor volume in groups 1-3 was similar and no statistical differences could be noted. On the contrary, when DOX and ZA were locally delivered using the micro-HA particles, a strong effect on tumor growth inhibition was observed and the tumor volume in group 4 was significantly lower than all other groups (p<0.01).

### DETAILED DESCRIPTION OF THE INVENTION

The inventors of present invention have surprisingly found that the combinations of particulate material and bisphosphonates and anthracyclinees result in an increased cell toxicity in respect of tumorous cells.

Thus, present invention relates to i.a. a composition comprising a particulate material, and further comprising at least two pharmaceutical drug agents, and wherein the drug agents are anti-cancer drugs and/or drugs used in treatment of cancer.

Present invention relates to a particulate material. The particulate material may in principle be any biocompatible material such as e.g. a material which may be absorbed and metabolised by the body.

In one aspect, the particulate material may be hydroxyapatite (HA) in any form or configuration. The term “particulate” is meant to be understood as a material present in a fine particles or particle sizes specified herein.

According to present invention, any type of suitable particulate material may be used. Suitable materials may be any material that is biocompatible, i.e. that may be implanted or otherwise administered to a subject in need.

One non-limiting example is hydroxyapatite. Hydroxyapatite (herein also, and interchangeably denoted as “HA”), is a naturally occurring mineral form of calcium apatite with the formula Ca₅(PO₄)₃(OH), but it is usually written Ca₁₀(PO₄)₆(OH)₂ to denote that the crystal unit cell comprises two entities.

The particulate material may be in any suitable size or size distribution. One non-limiting example is particles size ranges or distributions of e.g. about 1 nm to about 200 nanometers (nm), such as e.g. less than about 150 nm, less than about 100 nm, less than about 50 nm, less than about 45 nm, less than about 40 nm, less than 35 nm, less than about 30 nm, less than about 25 nm, less than about 20 nm, less than about 15 nm, or less than about 10 nm. In one aspect the particle size is in range of e.g. about 1 nm to about 100 nm.

In a further aspect, the particles may be about 1 nm, such as e.g. 5 nm, such as e.g. about 10 nm, such as e.g. about 15 nm, such as e.g. about 20 nm, such as e.g. about 25 nm, such as e.g. about 30 nm, such as e.g. about 35 nm, such as e.g. about 40 nm, such as e.g. about 45 nm, such as e.g. about 50 nm, such as e.g. about 100 nm, such as e.g. about 110 nm, such as e.g. about 150 nm, or such as e.g. about 200 nm.

In one aspect, the particle size may be in range of about 40 nm to about 150 nm.

In another aspect, the particle size may be in range of about 30 nm to about 70 nm, such as e.g. about 50 nm.

In another aspect, the particle size may be in range of about 20 nm to 80 nm, such as e.g. 30 nm to about 70 nm, or such as e.g. about 40 nm to about 60 nm, or such as e.g. about 20 nm, or about 30 nm, or about 40 nm, or about 50 nm, or about 60 nm, or about 70 nm, or about 80 nm. In a further aspect, the particle size may be about 50 nm.

In a further aspect, the particle size may be in range of about 80 nm to about 120 nm.

I another aspect, the particle size may be in range of about 100 nm to about 120 nm, or about 110 nm.

I a further aspect, the particle size may be in range of e.g. less than 200 µm, such as less than 100 µm, less than 50 µm, less than 35 µm, less than 20 µm or less than 10 µm. Moreover, the particles may be between about 0.1 and about 50 µm. In another aspect the microparticles may be in range of about 1 µm to about 500 µm, such as .e.g about 1 µm to about 100 µm, about 1 µm to about 50 µm, about 1 µm to about 25 µm, about 1 µm to about 15 µm, about 1 µm to about 10 µm, or about 1 µm, about 5 µm, about 10 µm, about 15 µm, about 20 µm, about 25 µm, about 30 µm, about 35 µm, about 40 µm, about 45 µm, about 50 µm, about 75 µm, about 100 µm, about 500 µm etc.

In one aspect, the particles may be in range of e.g. about 1 µm to about 10 µm.

In another aspect, the particles may be in range of e.g. about 5 µm to about 15 µm, such as e.g. about 10 µm.

In yet a further aspect, the invention relates to a mixture of particles, The mixture of particles may comprise particles in range of about 1 µm to about 500 µm, such as .e.g about 1 µm to about 100 µm, about 1 µm to about 50 µm, about 1 µm to about 25 µm, about 1 µm to about 15 µm, about 1 µm to about 10 µm, or about 1 µm, about 5 µm, about 10 µm, about 15 µm, about 20 µm, about 25 µm, about 30 µm, about 35 µm, about 40 µm, about 45 µm, about 50 µm, about 75 µm, about 100 µm, about 500 µm etc, and further comprise particles in range of e.g. about 1 nm to about 200 nanometers (nm), such as e.g. less than about 150 nm, less than about 100 nm, less than about 50 nm, less than about 45 nm, less than about 40 nm, less than 35 nm, less than about 30 nm, less than about 25 nm, less than about 20 nm, less than about 15 nm, or less than about 10 nm.

In one aspect, the invention relates to a mixture of particles in the range of e.g. about 1 µm to about 10 µm and in range of about 40 nm to about 100 nm, or e.g. about 80 nm to about 120 nm, or e.g. about 100 nm to about 120 nm, or about 110 nm.

In one aspect, the invention relates to HA particles that may be in range of e.g. about 1 µm to about 10 µm and in range of about 40 nm to about 100 nm, and HA particles of sizes of e.g. about 80 nm to about 120 nm, or e.g. about 100 nm to about 120 nm, or about 110 nm.

In yet a further aspect, the invention relates to HA particles that may be in range of e.g. about 1 µm to about 10 µm and in range of about 40 nm to about 100 nm, or e.g. about 80 nm to about 120 nm, or e.g. about 100 nm to about 120 nm, or about 110 nm, wherein any composition or composition being administered to a subject comprises HA particles in range of 1 µm to about 10 µm, and/or in range of about 40 nm to about 100 nm, or e.g. about 80 nm to about 120 nm, or e.g. about 100 nm to about 120 nm, or about 110 nm.

In one aspect, the HA particles consist or comprises particles in size range of 1 µm to about 10 µm, and/or in range of about 40 nm to about 100 nm, or e.g. about 80 nm to about 120 nm, or e.g. about 100 nm to about 120 nm, or about 110 nm.

In a further aspect, the HA particles consist or comprises particles in size range of 5 µm to about 15 µm, and/or in range of about 30 nm to about 70 nm.

Present invention relates to one or more anthracyclines.

In one aspect, the one or more anthracyclines may be e.g. doxorubicin, daunorubicin, epirubicin and idarubicin or any suitable mixtures thereof. Further non-limited examples are e.g. mitoxantrone or valrubicin.

In one aspect, the anthracycline may be e.g. doxorubicin.

Present invention also relates to one or more bisphosphonates.

In one aspect, the one or more bisphosphonates may be e.g. zoledronic acid, etidronic acid, clodronic acid, tiludronic acid, pamidronic acid, neridronic acid, olpadronic acid, alendronic acid, ibandronic acid, risendronic acid or any pharmaceutically acceptable salts thereof. The bisphosphonate be also be a salt wherein a radioactive compound is present or a radioactive compound as ⁹⁹Tc or ²²³Ra or strontium or samarium.

In one aspect, the bisphosphonate may be e.g. a nitrogen containing bisphosphonate such as e.g. alendronate, neridronate, ibandronate, pamidronate, risedronate, or zoledronic acid or any suitable pharmaceutically acceptable salts thereof.

In one aspect, the bisphosphonate may be e.g. zoledronic acid or any pharmaceutically acceptable salt or ester thereof.

In one aspect, the composition according to the invention may comprise a particulate material in size range of about 20 nm to about 100 nm, or e.g. about 80 nm to about 120 nm, or e.g. about 100 nm to about 120 nm, or about 20 nm, or about 30 nm, or about 40 nm, or about 50 nm, or about 60 nm, or about 70 nm, or about 80 nm, or about 90 nm, or about 100 nm or about 110 nm, and may further comprise doxorubicin and/or zoledronic acid or any pharmaceutically acceptable salt or ester thereof.

Consequently, and in one aspect, present invention relates to a composition comprising particulate hydroxyapatite in any configuration, one or more bisphosphonates and one or more anthracyclines.

In a further aspect, present invention relates to a composition comprising one or more nitrogen containing bisphosphonates, doxorubicin and a particulate material that may be e.g. hydroxyapatite.

As mentioned herein, present invention also relates to a novel method of administration and/or a novel method of treatment of a subject in need thereof.

In one aspect, the one or more pharmaceutical active compounds may be administered locally and/or systemically to a subject.

In one aspect, one pharmaceutical compound may be administered locally and the other systemically. In a further aspect, both pharmaceutical compounds may be administered systemically or locally.

In yet a further aspect, the one or more pharmaceutical compounds may be administered in any order or simultaneously.

The one or more pharmaceutical compounds may be administered by injection, or implantation, or alternatively in a tablet, capsule or any suitable formulation independently of each other. Alternatively, administration may be via the enteral route and/or the parenteral route.

In one aspect, present invention relates to treatment of any bone related tumour type such as e.g. osteosarcoma (OS) or also called osteogenic sarcoma (OGS).

In one aspect, the particulate material may be administered to a subject in need thereof. The administration may systemic or local, i.e. be administered to a desired site on the body of the subject. Thereafter, an anthracycline and/or bisphosphonate may be administered locally or systemically in any order, and either separately or simultaneously.

In a further aspect, the particulate material may be mixed with a bisphosphonate prior to being administered to a subject in need thereof. The administration may be systemic or local, i.e. be administered to a desired site on the body of the subject. Thereafter, a suitable anthracycline may be administered locally or systemically to the subject.

In yet a further aspect, the particulate material may be mixed with an anthracycline prior to being administered to a subject in need thereof. The administration may be systemic and local, i.e. be administered to a desired site on the body of the subject. Thereafter, a suitable bisphosphonate may be administered locally or systemically to the subject.

In another aspect, the particulate material may be mixed with an anthracycline and a bisphosphonate prior to being administered to a subject in need thereof. The administration may be systemic or local, i.e. be administered to a desired site on the body of the subject.

The composition according to the invention which may comprise either or all of HA particles, one or more anthracyclines, and one or more bisphosphonates in any combinations, and may be administered once daily, or such as e.g. once every two days, such as e.g. once every 24 h, or 2 days, 4 days, such as e.g. once every 7 days, or such as e.g. once every 14 days, or such as e.g. once every 28 days etc. The inventors of present invention has surprisingly found that the long term effect is especially pronounced. Already after 4 days, the long term effect is particularly noticed and even more so 7 days after administration of the composition according to the invention.

In one aspect, the invention relates to administration once every 7 days.

In yet a further aspect, the invention relates to administration of the composition according to the invention once every 4 days.

In yet a further aspect, the invention relates to administration of the composition according to the invention once every 48 h.

In yet a further aspect, the invention relates to administration of the composition according to the invention once every 24 h.

Present invention also relates to a compound for use in the treatment of one or more types of tumours, wherein the compound comprises a particulate material, one or more anthracyclines, and one or more bisphosphonates.

In one aspect, the compounds for use according to the invention may be for treatment of e.g. a bone related disease, such as e.g. one or more types of bone related tumours. In one aspect, the tumour may be e.g. osteosarcoma (OS) or also called osteogenic sarcoma (OGS).

In another aspect, the compounds for use according to the invention may be for the treatment of solid tumours of any kind.

In a further aspect, present invention relates to the compounds for use in the treatment of lung related cancers.

In yet another aspect, present invention also relates to the manufacture of a medicament for the treatment of cancer, wherein the medicament comprises a particulate material as disclosed herein and at least two pharmaceutical compounds as disclosed herein.

The at least two pharmaceutical compounds may be selected from one or more anthracyclines and one or more bisphosphonates. The particulate material may be e.g. hydroxyapatite.

As mentioned herein, it has surprisingly been found that a combination of a particulate material, one or more anthracyclines, and one or more bisphosphonates greatly improves treatment of various tumours and consequently an unexpected increase in tumour cell death. This leads to a more efficient treatment regimen.

### Experimental Section

Present invention will now be illustrated in the following non-limiting examples and shall not be construed as strictly limiting the invention in accordance with the example.

As used throughout the description, “HA” is intended to mean hydroxyapatite. “ZA” is intended to mean zoledronic acid or a suitable pharmaceutically acceptable salt thereof. “Dox” is intended to mean doxorubicin or a suitable pharmaceutically acceptable salt thereof.

### Example 1

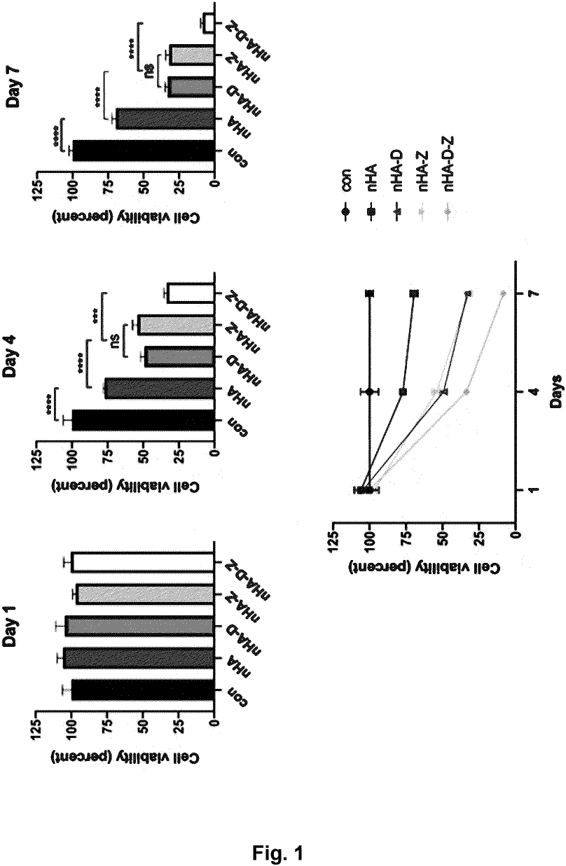

The inventors of present invention have surprisingly found that zoledronic acid (ZA) could increase the binding of doxorubicin (Dox) to nano-hydroxyapatite (nHA). In order to further investigate this, MG-63 cells were seeded in a 96-well plate and culturing for 24h to let cells attached to the bottom of the plate. The toxic effect of functionalized nHA was explored by setting up an experiment comprising thr MG-63 cells in 5 groups: 1. Control groups which was given normal medium; 2. Only nHA; 3. nHA-D (functionalized nHA with Dox); 4. nHA-Z (functionalized nHA with Dox); 5. nHA-D-Z (functionalized nHA with both Dox and ZA). Then check the cell viability on Day 1,4 and 7 after the point of administration. The result is illustrated in FIG. 1.

Preparation of functionalized nHA:
nHA: 10 mg nHA+250 µl PBS (average particle size of about 50 nm),
nHA-D: 10 mg nHA+100 ul Dox stock (170υg/ml)+150 µl PBS,
nHA-Z: 10 mg nHA+50 µl ZA stock (0.8 mg/ml)+200 µl PBS,
nHA-D-Z: 10 mg nHA+100 µl Dox stock (170 µg/ml)+50 µl ZA stock, (0.8 mg/ml)+100 µl PBS,

After mixing, leave them in a sonication bath for 15 mins, then on a shaker for 48 hrs. Centrifuge and throw the supernatant and wash the particles 4-5 times.

### Cell Culture and Treatment:

104 MG-63 cells were seeded in each well of 96-plate. After 24 hs, treatments were given. For all the particle group, nHA were given at the concentration of 100 µg/ml. Check cell viability on day 1, 4 and 7 by MTT cell proliferation/viability assay. As known to a person skilled in the art this is a colorimetric assay for assessing cell metabolic activity; MTT, a yellow tetrazole, is reduced to purple formazan in living cells. A solubilization solution like dimethyl sulfoxide (DMSO) is added to dissolve the insoluble purple formazan product into a colored solution. The absorbance of this colored solution can be quantified by measuring at a certain wavelength (usually between 500 and 600 nm) by a spectrophotometer. The degree of light absorption is dependent on the degree of formazan concentration accumulated inside the cell and on the cell surface, which could be used to measure cytotoxicity.

In more detail, we incubated MG-63 cells with different particles up to 7 days. The comparison was divided into 5 groups: 1, Control group without nHA; 2. Only nHA; 3. Functionalized nHA by Dox; 4. Functionalized nHA by ZA and 5. Functionalized by Dox and ZA. For all the particle groups, concentration of nHA was used as 100 µg/ml. We could see there is no toxic effect on day 1 for all the groups. On day 4, 22.9% cells were killed by nHA. And nHA-D or nHA-Z could lead to more cell death, with 50.7% and 45.9% respectively. nHA-D-Z has the best cytotoxicity with 66.4% cells dead. The similar trend of toxic effect from each group was also seen on day 7 with 66.9% cells dead in nHA-D group and 68% for nHA-Z. For nHA-D-Z group, almost 91.6% cells have been cleared. Based on these data, we surprisingly found that Dox or ZA could bind to nHA and functionalized nHA has a strong toxic effect on tumor cells. When loading both drugs on nHA, we could achieve the best cytotoxicity.

### Example 2

In a further experiment, a human lung cancer cell line A549 (adenocarcinomic human alveolar basal epithelial cells) was used as a model for lung cancer. Following treatment groups were used: 1. No treatment (negative control), 2) Nano-HA alone, 3) Nano-HA+ DOX (0.44 µg/well), 4) Nano-HA+ZA (0.08 µg/well) and 5) Nano-HA+ DOX (0.44 µg/well) + ZA (0.08 µg/well). HA particles had an average size of about 50 nm. In all treatment groups receiving the nano particles, a total of 20 µg/well HA particles were given in each well. 104 A549 cells were seeded in each well of a 96-well plate. Cells were cultured with RPMI 1640 medium+10% serum+1% antibiotics+1% glutamine. 24 h after seeding, medium was removed and new medium containing no nano particles (group 1) or nano particles alone or containing DOX or ZA or the combination (as described above in groups 2-5) were given to the cells. After a period of 48 h, the culture medium was replenished and new medium (similar to when the treatment was started) was provided to the cells for another 48 h. Finally, after a 4-day treatment period, viability of the cells was assessed using an MTT assay.

### Results:

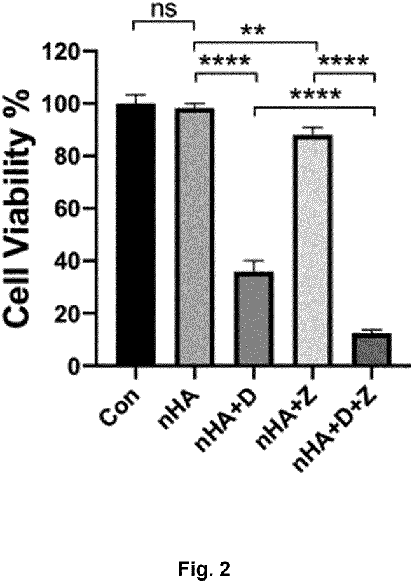

The viability of controls (no treatment) or nano-HA alone treated cells was almost 100% after 4-days of treatment. Addition of only ZA to the nano-HA particles led to only 10-15% reduction in cell viability. Addition of DOX to the nano-HA particles induced cell death in about 65% cells. However, the combination of DOX and ZA functionalized nano-HA was the most cytotoxic combination inducing apoptosis in nearly 85% of the cells. The results are illustrated in FIG. 2.

It can thus be concluded that a combination of hydroxyapatite particles, an anthracycline and a bisphosphonate results in an unexpected synergistic effect leading to an unexpected high cytotoxicity.

### Example 3

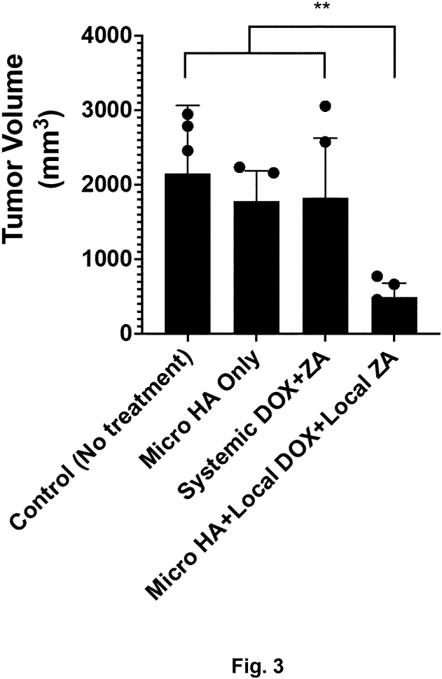

A human osteosarcoma model was created in Athymic nude mice using 4×10⁶ 143B human osteosarcoma cells injected sub-cutaneously. After a period of 7-days, when the tumor was palpable and clearly visible, a small skin incision was made and a sharp incision in the core of the tumor was created. After this, the tumor was treated as follows: 1) No treatment (n=5), 2) Micro Hydroxyapatite (HA) particles (size approximately 10 microns) (n=5), 3) Systemic delivery of Doxorubicin (DOX) and Zoledronic acid (ZA), given sub-cutaneously to each animal at doses of 60 µg and 10 µg, respectively (n=6) and 4) Micro HA particles combined with local DOX and ZA at doses of 60 µg and 10 µg, respectively (n=6). In groups 2 and 4, pellets of micro-HA with or without cytostatics were inserted into the core of the tumor. The incision was sutured and 14-days later, the effect of the treatment was evaluated by measuring the tumor volume using a Vernier-Caliper. The results of the experiment are illustrated in FIG. 3.

1-28. (canceled)

29. A composition comprising a particulate material, zoledronic acid or a pharmaceutically acceptable salt or ester thereof, and doxorubicin or a pharmaceutically acceptable salt or ester thereof,
wherein the particulate material is hydroxyapatite,
wherein the particulate material comprises particles in a size range of about 1 µm to about 20 µm or in a size range of about 20 nm to about 120 nm.

30. The composition according to claim 29, wherein at least one of the zoledronic acid or a pharmaceutically acceptable salt or ester thereof, or doxorubicin or a pharmaceutically acceptable salt or ester thereof is capable of binding to the particulate material.

31. The composition according to claim 29, wherein the particles of the particulate material comprise both particles in a size range of about 1 µm to about 20 µm and in a size range of about 20 nm to about 120 nm.

32. The composition according to claim 29, wherein the hydroxyapatite is present in particle size range of about 20 nm to about 80 nm.

33. A method of treating a bone-related cancer in a subject in need thereof, the method comprising administering to a composition according to claim 29 to the subject once once every 7 days.

34. The method according to claim 33, wherein the doxorubicin or a pharmaceutically acceptable salt or ester thereof is doxorubicin.

35. The method according to claim 34, wherein
(a) the doxorubicin is mixed with the particulate material prior to being administered to the subject, or
(b) the zoledronic acid or pharmaceutically acceptable salt or ester thereof is mixed with the particulate material prior to being administered to the subject, or
(c) both the doxorubicin and the zoledronic acid or pharmaceutically acceptable salt or ester thereof, is mixed with the particulate material prior to being administered to the subject.

36. The composition of claim 29, wherein the hydroxyapatite is in crystalline form.

37. The composition of claim 29, wherein the particles of the particulate material consist of particles in a size range of about 1 µm to about 20 µm and/or in a size range of about 20 nm to about 120 nm.

38. The composition of claim 32, wherein the particles of the particulate material have a size of about 50 nm.

39. The method of claim 33, wherein the bone-related cancer is osteosarcoma.
